(19) United States
(12) Patent Application Publication
(10) Pub. No.: US 2005/0014164 A1
(43) Pub. Date: Jan. 20, 2005

(54) Triplex-forming oligonucleotides containing modified purines and their applications

(76) Inventors: Ramon Eritja, Barcelona (ES); Anna Avino, Barcelona (ES)

Correspondence Address: Buchanan Ingersoll Professional Corporation;One Oxford Centre, 20th Floor, 301 Grant Street, Pittsburgh, PA, 15219-1410, US

(21) Appl. No.: 10/690,274

(22) Filed: Oct. 21, 2003

Related U.S. Application Data
(60) Provisional application No. 60/420,060, filed on Oct. 21, 2002.

(51) Int. Cl. C12Q 1/68; C07H 21/04

(52) U.S. Cl. 435/6; 536/23.1

(57) ABSTRACT

This invention presents oligonucleotide derivatives comprising a complementary purine part carrying one or more 8-aminopurines such as 8-aminoadenine, 8-aminoguanine and 8-aminohypoxanthine connected with a linker to an oligonucleotide carrying either GT or GA sequences. These oligonucleotide derivatives bind polypyrimidine sequences complementary (in the antiparallel sense) to the purine part by formation of purine-purine-pyrimidine triple helices. The oligonucleotides carrying 8-aminoguanines described in this invention have better binding properties than unmodified oligonucleotides. This enhancement in stability, coupled with the lack of an acidic pH requirement, makes the oligonucleotides carrying 8-aminopurines effective in applications involving oligonucleotide targeting of single stranded RNA in vitro and in vivo, as well as applications requiring triple helix formation.

### BENEFIT OF PRIOR PROVISIONAL APPLICATION

This utility patent application claims the benefit of co-pending U.S. Provisional Patent Application Ser. No. 60/420,060, filed Oct. 21, 2002, entitled “Triplex-forming Oligonucleotides Containing Modified Purines and Their Applications,” having the same named applicants as inventors, namely, Ramon Eritj a and Anna Avino. The entire contents of U.S. Provisional Patent Application Ser. No. 60/420,060 are incorporated by reference into this utility patent application.

### COMPUTER READABLE FORM AND SEQUENCE LISTING

This application contains a sequence listing in both written and computer readable form (CRF). The contents of the sequence listing information recorded in computer readable form as filed with this utility patent application is identical to the written (on paper) sequence listing as filed with this utility patent application.

### BACKGROUND OF THE INVENTION

1. Field of the Invention

This invention relates to an antiparallel oligonucleotide triplex having at least one modified purine. Oligonucleotide derivatives are provided having a first complementary purine carrying one or more 8-aminopurines connected with a linker to an oligonucleotide carrying GT or GA sequences. The oligonucleotide derivatives bind polypyrimadine sequences complementary (in the antiparallel sense) to the purine by formation of purine-purine-pyrimidine triple helix. Oligonucleotide hairpins and a method for stabilizing an antiparallel oligonucleotide triplex are also disclosed.

2. Description of the Background Art

Several years ago, it was shown that oligonucleotides could bind to homopurine-homopyrimidine sequences of double stranded DNA by forming triple helices. The formation of nucleic acid triple helices offers the possibility of designing sequence specific DNA binding molecules, which may have important uses as diagnostic tools as well as therapeutic treatments. For example, triple helices are used for the extraction and purification of specific nucleotide sequences, control of gene expression, mapping genomic DNA, detection of mutations of homopurine DNA sequences, site-directed mutagenesis, triplex-mediated inhibition of viral DNA integration, nonenzymatic ligation of double-helical DNA and quantitation of polymerase chain. However, one of the problems for the development of applications based on triple helix is the low stability of triple helices especially at neutral pH. To overcome this problem a lot of effort has been put into the design and preparation of modified oligonucleotides to enhance triple helix stability. One of the most successful modifications is to replace natural bases with some modified bases such as 5-methylcytidine, 5-bromouracil, 5-aminouracil, N⁴-spermine-5-methylcytidine, or 5-methyl-2,6(1H,3H)-pyrimidinedione.

Two different types of triple helices have been described. The purine: pyrimidine: pyrimidine motif or parallel triplex in which the purine: pyrimidine strands correspond to the target double-stranded DNA sequence (known as the Watson-Crick purine and pyrimidine strands) and the Hoogsteen strand is a pyrimidine strand used for the specific recognition of the double-stranded DNA. This motif is stable at acidic pH and less stable in neutral pH. This is due to the need of protonation of the cytosine at the Hoogsteen strand. The second motif is the so-called purine: purine: pyrimidine motif or antiparallel triplex in which the Hoogsteen strand is either a G, A-oligonucleotide or a G,T-oligonucleotide. This last type of triplex is less studied but it has more potential because the stability of this triplex is not dependent on pH.

Recent results have shown that the introduction of an amino group at position 8 of adenine increases the stability of triple helix due to the combined effect of the gain in one Hoogsteen purine-pyrimidine H-bond, and to the ability of the amino group to be integrated into the “spine of hydration” located in the minor-Major groove of the triplex structure. A similar behavior has been observed with 8-amino-2′-deoxyguanosine and 8-amino-2′-deoxyinosine. The preparation and the characterization of the binding properties of oligonucleotides containing 8-aminopurines has been described, but these oligonucleotides cannot be directly used for the specific recognition of double-stranded DNA sequences because the modified bases are purines that are in the target sequence and not in the Hoogsteen strand used for specific recognition of double-stranded DNA. Recently, it has been demonstrated that hairpins having a polypurine sequence with 8-aminopurines linked head-to-head with the Hoogsteen polypyrimidine sequence have a greater propensity than unmodified oligomers to form triplexes. The high degree of stabilization obtained with the addition of 8-aminopurines has been used for the development of new molecules to bind single-stranded nucleic acids by formation of parallel triplexes. In the present invention we describe the use of 8-aminopurines to obtain new molecules to bind single-stranded nucleic acids by formation of antiparallel triplexes.

The structure, dynamics and recognition properties of antiparallel DNA triplexes based on the antiparallel d(G-G•C), d(A-A•T) and d(T-A•T) motifs are studied by means of “state of the art” molecular dynamics simulations. Once the characteristics of the helix are defined, molecular dynamics and thermodynamic integration calculations are used to determine the expected stabilization of the antiparallel triplex due to the introduction of 8-aminopurines. Finally, oligonucleotides containing 8-aminopurine derivatives are synthesized and tested experimentally using different approaches in a variety of model systems. A very large stabilization of the triplex is found experimentally, as predicted by simulations. The results open a wide range of possibilities for the use of antiparallel triplexes in the context of antisense and antigene therapies.

The DNA is a largely polymorphic molecule, which in near-physiological conditions can adopt a variety of structures. Triple helices are one of these minor conformations of DNA which appear when a DNA duplex containing a polypurine track interacts with a third strand by means of specific H-bonds in the major groove of the duplex. The existence of DNA triple helices was theoretically suggested in 1953 by Pauling and Corey, and demonstrated experimentally by Rich and coworkers in 1957. Since then, triplexes have been the subject of a very intense research effort owing not only to its role in cellular life, but also to their possible biomedical (the anti-gene strategy) and biotechnological impact.

Depending on the orientation of the third strand with respect to the central polypurine Watson-Crick strand, the triplexes are classified into two main categories: i) parallel and ii) antiparallel. The parallel triplexes (also named pyrimidine-triplexes) are defined by three type of Hoogsteen triads (see FIG. 1): d(T-A•T), d(C-G•C) and d(G-G•C), where the first base refers to the Hoogsteen strand, and the symbols “dot•” and “dash-” refer to Watson-Crick and non-Watson-Crick pairings, respectively. The antiparallel triplexes (also named purine-triplexes) are based on three reverse-Hoogsteen triads: d(G-G•C), d(A-A•T) and d(T-A•T) (see FIG. 1).

Most structural studies on DNA triplexes have been focused on the parallel helices, which under normal laboratory conditions are more stable than the corresponding antiparallel structures. Accurate structural models of parallel triplexes have been derived from IR and NMR experiments, and molecular dynamics (MD) simulations. This large amount of information about the structure, reactive properties and flexibility of parallel triplexes allowed the design and synthesis of new molecules for the stabilization of the triplex in physiological conditions. Specially powerful are the 8-aminopurine derivatives of the invention, which are able to dramatically stabilize parallel triple helices based on the d(T-A•T) or d(C-G•C) triads.

A very scarce amount of high resolution structural information exists on the antiparallel triple helix. This is likely due to their reduced stability, which makes the structures stable only in the presence of a high concentration of divalent ions. However, despite their reduced stability under laboratory conditions, antiparallel triplexes seem to be more promising than the parallel ones in the biomedical field, since the formation of antiparallel triplex is pH independent, while that of parallel triplex requires in most cases an acidic pH, which does not always exist inside the cell. Accordingly, a clear need of more structural information of antiparallel triplexes exists, since this structural knowledge would help in the design of new strategies for the stabilization of this important family of triple helices.

### SUMMARY OF THE INVENTION

The present invention has met the hereinbefore described needs. The present invention provides triplex-forming oligonucleotide triplex comprising modified purines, wherein substitution of at least one purine in the triplex with at least one 8-aminopurine is set forth. The 8-aminopurine is preferably selected from the group consisting of 8-aminoadenine, 8-aminoguanine, and 8-aminohypoxanthine.

Another embodiment of this invention provides an oligonucleotide hairpin comprising a first oligonucleotide strand, a linker, and a second oligonucleotide strand, wherein the first oligonucleotide strand is a purine strand comprising at least one 8-aminopurine and the linker is connected to either the 3′ end of the first oligonucleotide strand and the 5′ end of the second oligonucleotide strand or to the 5′ end of the first oligonucleotide strand and the 3′ end of the second oligonucleotide strand.

In yet another embodiment, a method for stabilizing an antiparallel oligonucleotide complex is provided. This method comprises providing an antiparallel oligonucleotide triplex comprising a first, a second, and a third oligonucleotide strand, wherein at least one oligonucleotide strand comprises a purine, and replacing the purine with an 8-aminopurine.

The invention provides an antiparallel oligonucleotide triplex comprising a first oligonucleotide strand comprising at least one 8-aminopurine, a linker connected to the first oligonucleotide strand, and a second oligonucleotide strand connected to the opposite end of the linker from the first oligonucleotide strand and capable of forming a hairpin with the first oligonucleotide strand, and a third oligonucleotide strand comprising pyrimidines, wherein the third oligonucleotide strand is substantially complementary to and antiparallel to the first oligonucleotide strand.

This invention presents analysis of novel antiparallel triplexes using state of the art MD simulations. Structures based on the d(G-G•C), d(A-A•T) and d(T-A•T) triads were analyzed using nanosecond-time scale simulations in aqueous solvent. The equilibrated structures were used to study the impact of 8-aminopurine substitutions in the stability of the different antiparallel triplexes by means of a combination of MD and thermodynamic integration (TI) simulations. Finally, the 8-aminopurine derivatives were synthesized and for the first time incorporated into antiparallel triplexes. Thermodynamic and gel retardation experiments allowed us to confirm experimentally that 8-aminopurine derivatives lead to extremely stable antiparallel triplexes, even at conditions mimicking the physiological ones. The impact of this discovery in the design of new antigene and antisense strategies is discussed.

### BRIEF DESCRIPTION OF THE FIGURES

FIG. 1. Schematic representation of different triads found in triplexes. First row: Hoogsteen pairings found in parallel triplexes. Second row: reverse-Hoogsteen pairings found in antiparallel triplexes. Third row: suggested reverse-Hoogsteen pairings involving 8-aminoadenine and 8-aminoguanine. The position of the minor (m), minor-Major (mM) and Major-Major (MM) grooves is displayed for each triad.

FIG. 2. Root mean square deviations (RMSd in Å) between the structures of the 10-mer triplexes sampled during trajectories and reference conformations.

FIG. 3. Schematic representation of the MD-averaged structure of the 10-mer antiparallel triplexes (T1 to T4 from left to right) studied here.

FIG. 4. Distribution plots corresponding to: A. Phase angle of the 2′ deoxyriboses corresponding to all nucleotides, those located in the Watson-Crick strands, and those located in the Hoogsteen strand. B. Selected helical parameters. Values were obtained by collecting data along the trajectories. The range of values found in NMR structures (entries pdb134d and pdb135d) is displayed as lines (solid and dot-dashed respectively) in the plots.

FIG. 5. MIP (TOP) and solvation (BOTTOM) maps corresponding to triplexes T1 to T4 (from left to right). MIP contour correspond to interaction energies of −7 kcal/mol. Solvation maps correspond to a density of water of 3.5 g/cm³.

FIG. 6. Photograph of a 15% polyacrylamide gel containing 90 mM TB (pH 8.0), 50 mM MgCl₂ and differing stoichiometric ratios of d(C₃T₄C₃) (pyr) (SEQ ID NO: 12) and d(GG^{N}G^{N}A₄G^{N}G^{N}G) (pur) (SEQ ID NO: 14) stained with stains-all. Lane A: Duplex refers to d(C₃T₄C₃). d(GG^{N}G^{N}A₄G^{N}G^{N}G) (SEQ ID NO: 12, SEQ ID NO: 14), and triplex refers to d(C₃T₄C₃).2[d(GG^{N}G^{N}A₄G^{N}G^{N}G)] (SEQ ID NO: 12, SEQ ID NO: 14, SEQ ID NO: 14) BPB: bromophenol blue.

FIG. 7. Hypothetical base-pairing schemes of triads of antiparallel triplexes containing 8-aminopurines.

FIG. 8. Schematic representation of the reverse Hoogsteen d(G-G•C) triad, and those formed by inosine (I) and 8-aminoinosine (I^{N}).

FIG. 9. Exchangeable proton NMR spectra of the triplex formed by H26GT (SEQ ID NO: 16), H26GT2AG (SEQ ID NO: 20) with their polypyrimidine target WC-11mer (SEQ ID NO: 15).

### DETAILED DESCRIPTION OF THE INVENTION

Molecular Dynamics Simulations

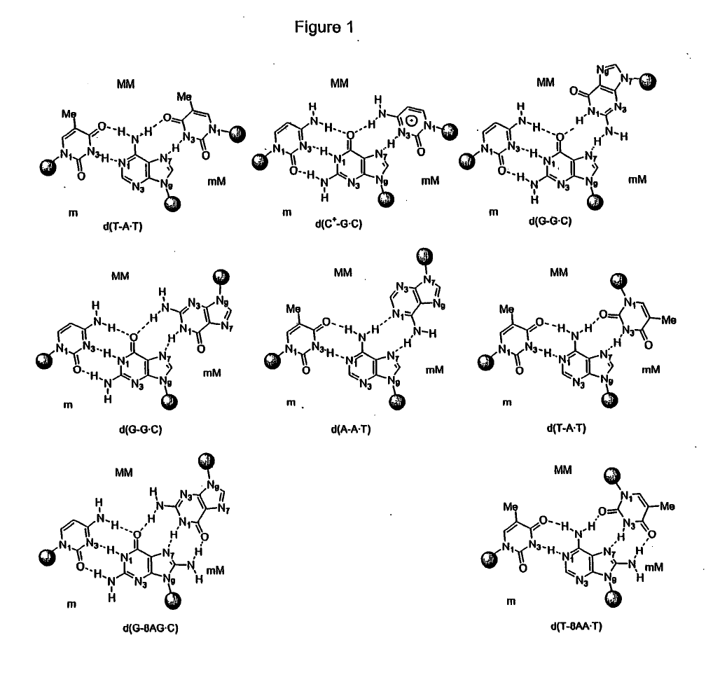

MD simulations were performed to analyze the structural, dynamic and recognition properties of antiparallel duplexes containing all type of antiparallel triads (see FIG. 1). For this purpose starting models of the following triplexes were generated (see Table 1): i) a 10-mer polyd(G-G•C) triplex (named T1 in the paper), (SEQ ID NO: 1, SEQ ID NO: 2, SEQ ID NO: 1) ii) a 10-mer polyd(A-A•T) triplex (T2) (SEQ ID NO: 4, SEQ ID NO: 3, SEQ ID NO: 4), and iii) a 10-mer poly(T-A•T) triplex (T3) (SEQ ID NO: 3, SEQ ID NO: 3, SEQ ID NO: 4), and iv) a 10-mer triplex containing both d(T-T•A) and d(G-G•C) triads with the reverse Hoogsteen pairing (T4 in Table 1) (SEQ ID NO: 6, SEQ ID NO: 7, SEQ ID NO: 8). In order to analyze the impact of the oligonucleotide size in the structure of the triplexes additional simulations were performed for: iv) a 8-mer d(A-A•T) triplex (T5), v) a 9-mer polyd(T-A•T) triplex (T6), and vi) two 7-mer triplex containing both d(T-T•A) and d(G-G•C) triads with the reverse Hoogsteen pairing (T7a and T7b simulations in Table 1).

Starting structures for triplexes T1-T7a were generated from Patel's structure of the triplex d(AGGAGGA) containing d(A-A•T) and d(G-G•C) triads (PDB entry pdb134d). Sequences were modified when needed, and triplexes longer than 7 triads were extended using average helical parameters. For comparison, one simulation (T7b) was repeated using as starting conformation another triplex structure deposited also by Patel's group in PDB as entry pdb135d (all atoms RMSd between the two NMR structures is 0.9 Å (for the duplex portion) and 1.2 Å (for the entire molecule)). This new trajectory was very similar to that found for T7, demonstrating that MD simulations are not very dependent on small structural changes in the starting configuration. All the structures were partially minimized to avoid incorrect geometries for 1000 steepest descent and 1000 conjugate gradient cycles. These relaxed systems were then surrounded by water (between 2200 and 2800 TIP3P (molecules) and Na⁺ to achieve neutrality. These systems were then optimized, thermalized and equilibrated using our standard multistage protocol which extends for 200 ps. Equilibrated systems were then subject to production runs of 5 ns (T1), 3 ns (T2-T4) and 2 ns (T5-T7) of unrestrained MD simulation.

To analyze the structural impact of the introduction of 8-aminopurines according to the present invention, we defined additional triplexes containing these derivatives in at least one position of the triplex (see Table 1). Structures were created from the corresponding reference triplexes (see above), and were then hydrated, optimized and equilibrated using a protocol identical to that described above. Simulations of triplexes containing 8-aminopurines were extended to 2 ns in all the cases. Finally, when needed for thermodynamic integration simulations (see above) Watson-Crick duplexes were generated using standard fiber parameter, hydrated, neutralized, optimized, heated and equilibrated as noted above. Unrestrained simulations for duplexes (both modified and unmodified) extend for 2 ns.

All the MD simulations were carried out in the isothermic-isobaric ensemble using periodic boundary conditions and the particle mesh-Ewald technique to represent long-range electrostatic effects. AMBER-99 and TIP3P force-fields were used to represent molecular interactions in the system. Parameters for 9-aminopurines were taken from our previous parametrization works. SHAKE was used to constrain all the bonds at their equilibrium distances, which allowed us to use 2 fs time scale for integration of Newton's laws of motion. The AMBER-6.0 computer program was used for all MD simulations. Simulations reported here correspond to up to 30 ns of unrestrained MD simulation of antiparallel triplexes (22 ns of unmodified triplexes, and 8 ns of triplexes containing 8-aminopurine derivatives). This is to our knowledge the most complete theoretical study of antiparallel triplexes published to date.

Energetic analysis of the trajectories was carried out using analysis modules in AMBER6.0, as well as “in house” programs. Interaction maps were determined using our cMIP methodology and considering a O⁺ probe molecule. Hydration maps were obtained by integrating solvent population during the dynamics using “in house” programs.

Free Energy Calculations

MD/TI calculations were performed to determine the gain in stability of antiparallel triplexes obtained by the substitution of purines by 8-aminopurines. For this purpose, and following standard thermodynamic cycles, mutations between 8-aminopurines and purines were performed in different triplexes and duplexes. Mutations between oligonucleotides containing 8-aminoguanine (G^{N}) and 8-aminoadenine (A^{N}) and those containing parent nucleobases were performed using 21 or 41 windows of 10 ps each (independent free energy estimates were taken from the first and second halves of each window), leading to simulations of 420 and 840 ps. To gain statistical confidence in the results, mutations were carried out in both 8-aminopurine→purine and purine→8-aminopurine directions. This means that each free energy difference value reported in the paper was obtained by averaging 8 independent estimates of the same process. Structures for the different oligonucleotides containing 8-aminopurines were built from MD-averaged structures of the corresponding unmodified oligonucleotides, and equilibrated for 2 ns of unrestrained MD. In all the cases the mutations were done in positions located in the center of the helix

The impact of the 8-aminopurine substitution on the triplex stability was determined as the difference between the free energy associated to the mutation in the triplex and in the duplex (see equation 1). That is, MD/TI calculations provide a direct estimate of the change in the free energy of the triplex→duplex transition associated to the substitution of a purine (in the Watson-Crick position) to 8-aminopurine.
ΔΔG(Y→Y^{N})=ΔG(Y→Y^{N})ₜᵣᵢₚₗₑₓ−ΔG(Y→Y^{N})_{duplex} (1)

Oligonucleotide Synthesis

Oligonucleotides were prepared on an automatic Applied Biosystems 392 DNA synthesizer. The phosphoramidites of 8-aminoadenine, 8-aminoguanine and 8-aminohypoxanthine were prepared using techniques well known by those skilled in the art. The phosphoramidite of protected 8-amino-2′-deoxyinosine was dissolved in dry dichloromethane to yield a 0.1 M solution. The remaining phosphoramidites were dissolved in dry acetonitrile (0.1 M solution). Oligonucleotides containing natural bases were prepared using commercially available chemicals and following standard protocols. After the assembly of the sequences, oligonucleotide-supports were treated with 32% aqueous ammonia at 55° C. for 16 h (hour) except for oligonucleotides bearing 8-aminoguanine. In this case, a 0.1 M 2-mercaptoethanol solution in 32% aqueous ammonia was used and the treatment was extended to 24 h at 55° C. Ammonia solutions were concentrated to dryness and the products were purified by reversed-phase HPLC. Oligonucleotides were synthesized on a 0.2 μmol scale and with the last DMT group at the 5′ end (DMT on protocol) to facilitate reversed-phase purification. All purified products presented a major peak, which was collected. Yields (OD units at 260 nm after HPLC purification, 0.2 μmol) were between 5-10 OD. HPLC conditions: HPLC solutions were as follows. Solvent A: 5% ACN in 100 mM triethylammonium acetate pH 6.5 and solvent B: 70% ACN in 100 mM triethylammonium acetate pH 6.5. Columns: PRP-1 (Hamilton), 250×10 mm. Flow rate: 3 ml/min. A 30 min linear gradient from 10-80% B (DMT on) or a 30 min linear gradient from 0-50% B (DMT off).

PAGE Retardation Assays

Nondenaturing polyacrylamide gel electrophoresis was carried out at 4° C. The 15% polyacrylamide gels [29:1 acrylamide:bis(acrylamide)] contained 90 mM Tris-acetate (TB) pH 8.0 and 50 mM MgCl2. All DNA samples were preheated at 90° C. for 5 min, slowly cooled, and loaded in 90 mM TB pH 8.0, and 50 mM MgCl2, 5% glycerol, containing bromophenol blue (BPB) and xylene cyanol (XC) dyes. Gels were stained for 20 min in a 0.1 mg/ml solution of stains-all in 15% formamide in water, briefly washed with distilled water, destained with a IR lamp and photographed.

Helix-Coil Transitions and Thermodynamic Analysis.

Melting experiments with duplex d(C₃T₄C₃).d(GG^{N}G^{N}A₄G^{N}G^{N}G) (SEQ ID NO: 12, SEQ ID NO: 14) and triplex d(C₃T₄C₃).2[d(GG^{N}G^{N}A₄G^{N}G^{N}G)] (SEQ ID NO: 12, SEQ ID NO: 14, SEQ ID NO: 14) were performed as described by Pilch et al., as known by those skilled in the art.

Melting experiments with triple helices were performed as follows. Solutions of equimolar amounts of hairpins and the target Watson-Crick pyrimidine strand (11-mer) (SEQ ID NO: 15) were mixed in 10 mM sodium cacodylate, 50 mM MgCl₂ and 0.1 mM EDTA at pH 7.2. The DNA concentration was determined by UV absorbance measurements (260 nm) at 90° C., using for the DNA coil state the following extinction coefficients: 7500, 8500, 12500, 12500, 15000 and, 15000 M⁻¹ cm⁻¹ for C, T, G, 8-amino-G, A and, 8-amino-A, respectively. The solutions were heated to 90° C., allowed to cool slowly to room temperature, and stored at 4° C. until UV was measured. UV absorption spectra and melting experiments (absorbance vs temperature) were recorded in 1 cm path-length cells using a spectrophotometer, with a temperature controller and a programmed temperature increase rate of 0.5° C./min. Melts were run on triplex concentration of 3 μM (1-1.2 O.D. units at 260 nm).

The samples used for the thermodynamic studies were prepared in a similar way, but melting experiments were recorded at 260 nm and using 0.1, 0.5 and 1 cm path-length cells.

Thermodynamic data were analysed as described elsewhere. Melting curves were obtained at concentrations ranging from 0.5 to 20 μM of triplex. The melting temperatures (Tm) were measured at the maximum of the first derivative of the melting curve. The plot of 1/Tm versus lnC was linear. Linear regression of the data gave the slope and the y-intercept, from which ΔH, and ΔS were obtained. The free energy was obtained from the standard equation: ΔG=ΔH−TΔS.

Structural Description of the Antiparallel Triplex Helix

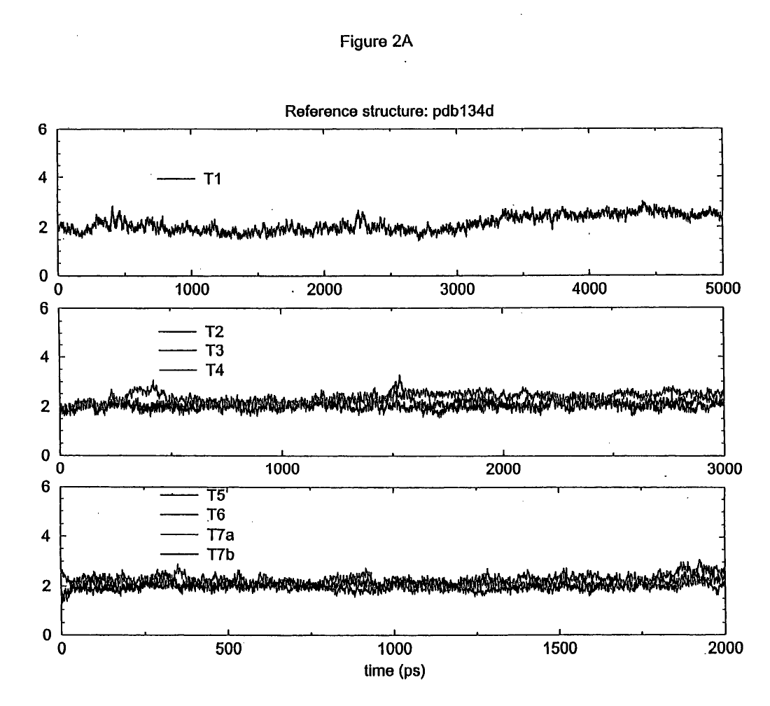

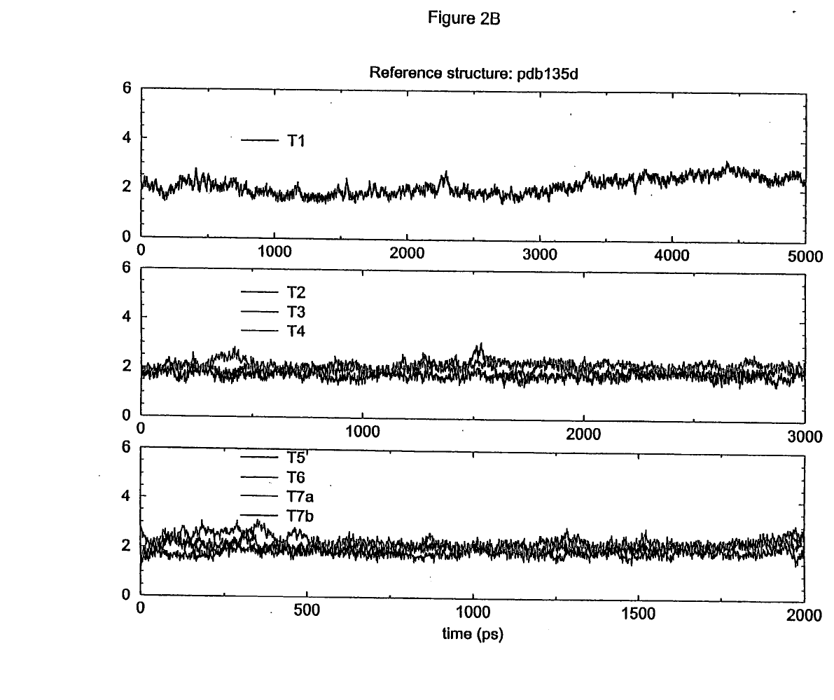

Equilibrium trajectories of the different triplexes considered here seem well equilibrated, as noted in FIG. 2, and in Table 2. The RMSd with respect to the respective MD-averaged structures are very small (around 1 Å) in all the cases, indicating that the trajectories are visiting a quite well defined region of the configurational space, and that the flexibility of the triplex is quite reduced compared with that of duplexes with the same (Watson-Crick) sequence. The root mean square deviations (RMSd) with respect to the NMR structures in pdb134d and pdb135d (computed using all backbone atoms of the central 7-mer sequence) are also quite small around 2 Å. For the T7a and T7b triplexes (those corresponding to the same oligonucleotides studied by Patel's group) the RMSds between theoretical and NMR structures are around 2.2 Å (only backbone atoms) and 1.8 (all atoms), confirming that the MD trajectories are sampling similar regions of the configurational space than those detected in the NMR experiments. Considering the ability of MD simulations to escape from incorrect triplex conformations, the agreement between MD and NMR results cannot be considered fortuitous or related to incomplete sampling of the MD trajectory.

Analysis of the different trajectories in Table 2 and FIG. 2 illustrates the independence of the results on the length of the oligonucleotide (in the range 7-10 mer), on the starting conformation for the simulation (pdb134d or pdb135d), and on the length of the trajectory (in the 1-5 ns range). The introduction of one or two 8-aminopurines leads to negligible changes in the structures, as found previously for parallel triplexes. In all the cases studied here the RMSds between the oligonucleotides containing 8-aminopurines and the parent oligonucleotides are below 1 Å (i.e. very close to the thermal noise of the simulation). In summary, the main characteristics of the antiparallel triplex seem well defined and independent on the definition of the simulation model, and of the presence small chemical alterations in the structure. We can then safely discuss the characteristics of the antiparallel triplexes containing the three reverse Hoogsteen triads: d(T-A•T), d(G-G•C) and d(A-A•T) by analyzing only simulations for the larger unmodified oligonucleotides T1-T4.

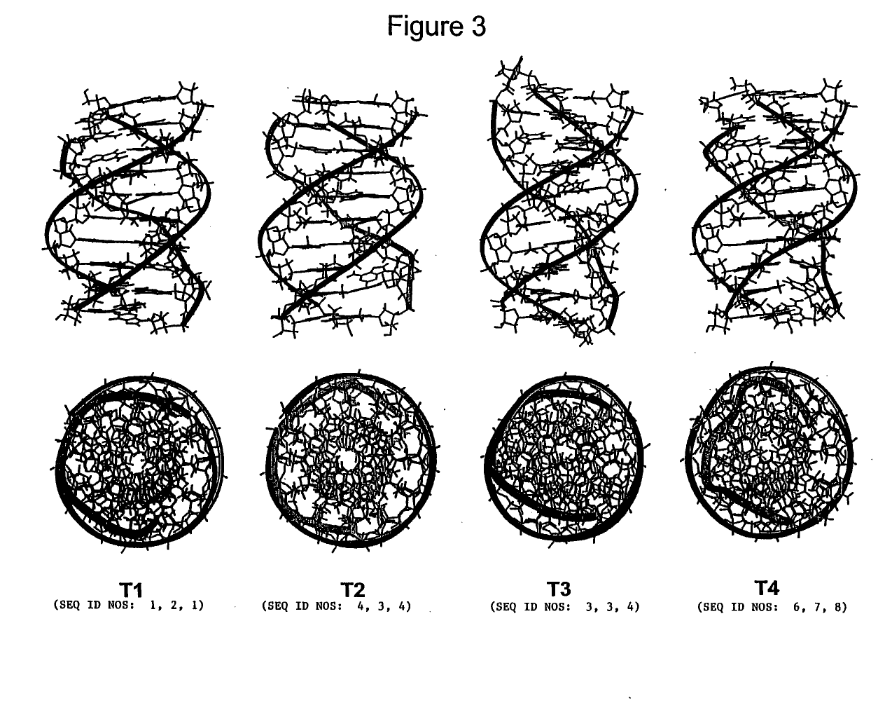

As suggested by the RMSd profiles (FIG. 2) the general shape of the helices is well preserved during all the simulations (FIG. 3), despite no negligible distortions in the Hoogsteen strand of sequences containing adjacent d(T-A•T) triads (T3 and T4). The Watson-Crick hydrogen bonds are present during 98-100% of the time for trajectories T1-T4 (the same values are obtained for simulations T5-T7), but the reverse-Hoogsteen hydrogen bonds are more labile, specially for adjacent d(T-A•T) triads, as noted in the fact that around 27% of the simulation T3 and 32% of simulation T4 show disruption of T-A reverse Hoogsteen hydrogen bonds, due to breathing movements. Similar values were obtained for T6, while greater conservation of reverse Hoogsteen hydrogen bonds (86% and 92%) is found for triplexes T7a and T7b, which contain d(T-A•T) triads, but not in contiguous positions. Analysis of trajectories T3, T4, and T6 show that the partial disruption of reverse Hoogsteen hydrogen bonds is due to a clear tendency of the reverse Hoogsteen thymine in polyd(T-A•T) tracks to escape from the planarity of the Watson-Crick d(A•T) pair in a pseudo-propeller twist movement. The lost or reverse Hoogsteen hydrogen bonds is less important for triplexes containing only d(G-G•C) and d(A-A•T) triads (85% and 90% of reverse Hoogsteen hydrogen bonds are present in simulations T1 and T2). Interestingly, the magnitude of breathing in antiparallel d(T-A•T) triplexes is much larger than that found for the parallel d(T-A•T) structures. This finding suggests that the parallel arrangement is clearly more stable for the d(T-A•T) triads, despite the similar stability of Hoogsteen and reverse Hoogsteen hydrogen bonds.

The general structure of antiparallel triplexes is surprisingly similar to that of parallel triplexes. This is noted in RMSds in the range 1-2 Å between the Watson-Crick backbones of the MD-averaged parallel and antiparallel triplexes (see Table 3). Interestingly, the general characteristics of the antiparallel triplex are quite independent of the sequence, as noted in RMSds also in the range 1-2 Å between the Watson-Crick backbones of MD-averaged structures of simulations T1, T2, T3 and T4 (see Table 3). However, the introduction of the third strand in the calculation of the RMSd leads to a dramatic increase of c.a. 1 Å, confirming that the largest sequence-dependent changes are always located in the third strand, while the core of the triplex (defined by the Watson-Crick strands) is less sensitive to sequence effects. Cross RMSd relationships in Table 3 suggests that triplexes containing only d(G-G•C) and/or d(A-A•T) triads (T1,T2) are very similar, and both are slightly different to triplexes containing d(T-A•T) triads (T3,T4), which appear more distorted from an ideal helical conformation. The reduced stability of the d(T-A•T) reverse Hoogsteen triads can explain the differential structural properties of triplexes containing this type of triads. Additional distortions in the third strand found in T4 (see FIG. 3) are probably due to the different size of d(T-A•T) and d(G-G•C) triads.

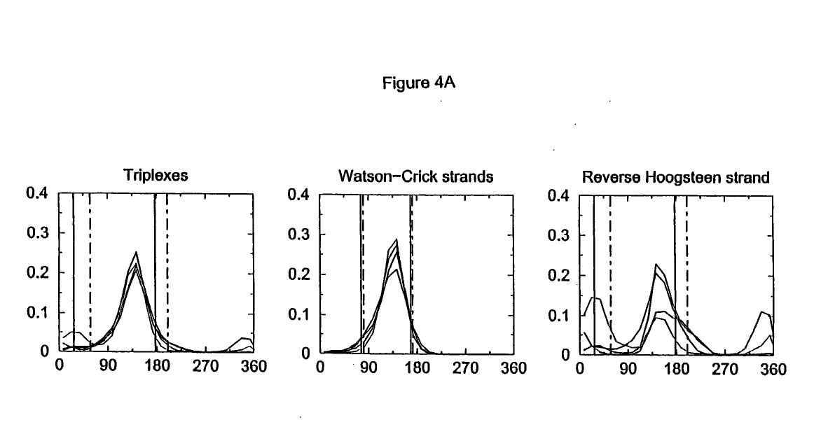

The helical analysis of triplexes T1-T4 show quite standard values for triplexes, with average twist around 30 degrees (it increases to 32 degrees for T3), rise values around 3.4 Å, small inclination, roll and propeller twist, and x-displacement values small (in absolute terms) and negative (see Table 4). In average the sugars are in the South to South-East regions (see Table 4 and FIG. 4A) for all the triplexes, as expected for structures pertaining to the B-family. However, large differences exist in the puckering population between Watson-Crick and reverse Hoogsteen strands (see FIG. 4A). Thus, the vast majorities of sugars in the Watson-Crick strands are found with phase angles in the range 90-180 degrees. On the contrary, large population of North puckerings are found in the reverse Hoogsteen strand, and in fact, for one of the triplexes (T3) North puckerings are more populated than South puckerings. In summary, as found in previous works with parallel triplexes, the sugars of the Watson-Crick are restricted to be in the South-South East region of the pseudorotational circle, but sugars in the third strand are more free to move and sample North regions.

As noted in the standard deviations in Table 4 and in distribution plots in FIG. 4 the triplexes display a non-negligible flexibility and sample wide regions of the helical space during the trajectories. It is worth noting that all the values found during the trajectories fall within the range of variability found in NMR structures (see Table 4 and FIG. 4) suggesting that helical parameters obtained from unrestrained trajectories obtained here are consistent with experimental NMR data. Clearly, the agreement is even better if NMR structure is compared with results of trajectories T7a and T7b. Finally, it is again clear from Table 4 and FIG. 4 that despite the general similarity between the four triplexes, no negligible alterations in the structure are originated by the presence of unstable d(T-A•T) reverse Hoogsteen triads, which when present adjacent in the sequence introduce distortions in the third strand, and by the lack of isomorphism of d(G-G•C) and d(T-A•T) triads.

Interestingly, most helical values in Table 4 mirror the values obtained for parallel triplexes, a fact that it is not surprising considering the small RMSd between parallel and antiparallel triplexes. The only major differences between parallel and antiparallel triplexes are found in the grooves (see Table 4). For parallel triplexes the presence of the poly-pyrimidine Hoogsteen strand breaks the major groove of the duplex in two asymmetric grooves (see FIG. 1 for nomenclature): the minor part of the major groove (mM) and the major part of the major groove (MM). For a parallel d(T-A•T) triplex the width (measured as the shortest P-P distance) of the grooves are around 17 Å (MM), 12 Å (m), and 9 Å (mM), that is the partition of the major groove by the Hoogsteen strand is very asymmetric leading to a very narrow mM groove and a very wide MM groove, which can be large enough to interact with proteins. The situation is completely different for the antiparallel triplexes studied here, where the presence of the reverse Hoogsteen strand breaks more symmetrically the major groove of the duplex. Thus, the MM groove is very flexible, but in average is only 1 Å wider than the mM groove, compared to the large difference (8 Å) found parallel triplexes. The average width of the minor groove is around 12 Å, a value similar to that obtained for parallel triplexes, and for normal B-DNA duplexes, showing that the minor groove is not dramatically altered by the presence of the third strand. The effect of the sequence in the width of the groove is moderate, and implies a reduction in the width of the m-groove and a parallel increase in the width of the mM one for triplexes containing the d(T-A•T) triad.

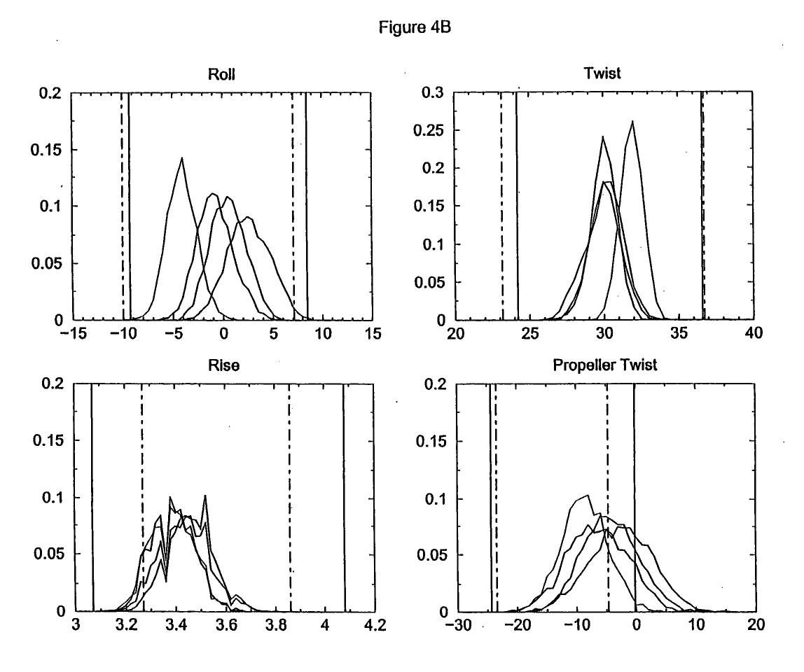

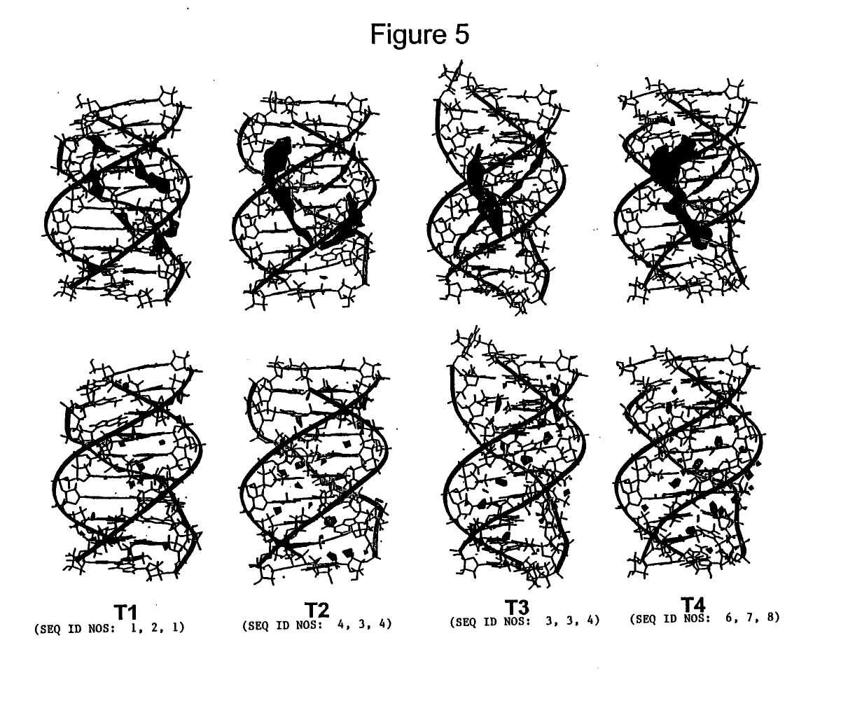

A deeper insight on the characteristics of the grooves in the antiparallel triplexes is obtained by inspection of MIP maps in FIG. 5. MIP analysis show that the potentiality for interaction of cations with the triplex depends on the sequence. For triplex T1 (SEQ ID NO: 1, SEQ ID NO: 2, SEQ ID NO: 1), the regions of better interaction are located in the MM and mM grooves, while the m-groove is not such a good target due probably to the presence of the 2-amino groups of guanines, as found in DNA duplexes. Triplex T2 (SEQ ID NO: 4, SEQ ID NO: 3, SEQ ID NO: 4) shows favorable regions of interaction in the three grooves, the MM groove being the best target for cationic interaction. Finally, triplexes T3 (SEQ ID NO: 3, SEQ ID NO: 3, SEQ ID NO: 4) and T4 (SEQ ID NO: 6, SEQ ID NO: 7, SEQ ID NO: 8) show a marked region of favorable interaction in the MM groove, and another region of favorable interaction located in the m-groove at steps containing d(T-A•T) triads. A simple inspection of H-bond donors and acceptors in the three grooves (see FIG. 1) helps to rationalize the MIP profiles.

Density maps provide a MD-averaged picture of the ability of the different triplexes to interact with water. All the triplexes are well hydrated, with extended regions where the density of water is 3.5 times above the background of the simulation (see FIG. 5). Clear spines of hydration are found for all the triplexes located in the m-groove (see FIG. 5). Such spines are not disrupted in the presence of d(G-G•C) triads, despite the perturbing effect of the 2-amino group of the Watson-Crick guanines. Additional strands of water are found in the mM groove (more clear in triplexes T3 (SEQ ID NO: 3, SEQ ID NO: 3, SEQ ID NO: 4) and T4 (SEQ ID NO: 6, SEQ ID NO: 7, SEQ ID NO: 8)), and small strands of water are also located in the MM groove of the four triplexes. In summary, the water seems to take advantage of its ability to act as H-bond donor and acceptor and is able to interact very well with all the triplex structures. Overall, the patterns of hydration found here are not very different to those previously obtained for parallel triplexes, despite of the different size of the grooves in parallel and antiparallel triplexes.

Free Energy Calculations

The substitution of adenine by 8-aminoadenine and guanine by 8-aminoguanine strongly stabilizes antiparallel triplexes. Such stabilization is justified by the gain of a strong Hoogsteen hydrogen bond, and by the insertion of the 8-amino group in the mM groove replacing a water molecule with a net entropic gain. Inspection of FIGS. 1 and 5 suggests that substitution of the Watson-Crick guanine by 8-amino guanine stabilizes the d(G-G•C) triad, and simultaneously the substitution of the Watson-Crick adenine by 8-amino adenine stabilizes the d(T-A•T) triad. Models of triplexes containing 8-amino derivatives were generated and equilibrated using MD protocols (see Table 1). Comparison of the corresponding trajectories (T1n (SEQ ID NO: 1, SEQ ID NO: 2, SEQ ID NO: 5), T1nn (SEQ ID NO: 5, SEQ ID NO: 2, SEQ ID NO: 5), T4n (SEQ ID NO: 6, SEQ ID NO: 7, SEQ ID NO: 9) and T6n), with the parent trajectories TI (SEQ ID NO: 1, SEQ ID NO: 2, SEQ ID NO: 1), T4 (SEQ ID NO: 6, SEQ ID NO: 7, SEQ ID NO: 8) and T6 shows that the structural impact of the presence of 8-amino purines in the triplexes is very small, and localized in the substituted triad, which becomes more planar in the presence of 8-amino purines. The substitution of the Watson-Crick guanine (d(G-G•C) triad) by 8-amino guanine and the substitution of adenine (d(T-A•T) triad) by 8-amino adenine leads to an improvement of nearly 6 kcal/mol in the reverse-Hoogsteen hydrogen bonding energy, with little (0.4-0.1 kcal/mol) impact in the stacking energy and Watson-Crick hydrogen bonding (around −0.2 kcal/mol). That is to say, basic energy considerations suggest that the introduction of 8-aminopurine derivatives stabilize triplex helices containing the d(G-G•C) or d(T-A•T) triads.

In order to obtain a more quantitative prediction of the impact of the substitution of purines by 8-amino purine we perform MD/TI simulations in which the works associated to the mutations A←→A^{N} and G←→G^{N} were computed, which implies performing 4 mutations for each triplex and duplex (see Table 5). The mutation profiles are smooth without clear discontinuities, suggesting the lack of hysteresis effects, and the standard errors in the free energy estimates are very small (0.2-0.4 kcal/mol), suggesting good convergence in the results.

Clearly, results in Table 5 strongly suggest that the presence of a single 8-amino purine strongly stabilizes triplexes based on both d(G-G•C) and d(A-A•T) triads. Interestingly, results for triplex T1n (SEQ ID NO: 1, SEQ ID NO: 2, SEQ ID NO: 1) and T1nn (SEQ ID NO: 5, SEQ ID NO: 2, SEQ ID NO: 5) are similar, which suggests that the presence of G^{N} in the Hoogsteen strands (as in some of the experimental models used in this work) does not alter the triplex stabilizing effect of G^{N} in the Watson-Crick position. Very interestingly, the gains in antiparallel triplex stability induced by the 8-aminopurines are very similar to those induced by the same molecules in parallel triplexes, suggesting that the introduction of 8-aminopurines in the Watson Crick strand is a very strong, and nearly universal mechanism of triplex stabilization. As in antiparallel triplexes, the gain in H-bonding and the entropy gain related to the liberation of waters in the mM groove appear as the main factor responsible for the gain in stability of the triplexes induced by the presence of G^{N} or A^{N} in the Watson-Crick position.

Experimental Studies of the Stability of a Short Intermolecular Antiparallel Triplex Containing 8-aminoguanines.

MD and MD/TI calculations show that G→G^{N} substitutions stabilize antiparallel triplexes containing the d(G-G•C) triad. Oligonucleotides were prepared carrying 8-aminopurines and then the triplex-forming properties of these oligonucleotides were studied.

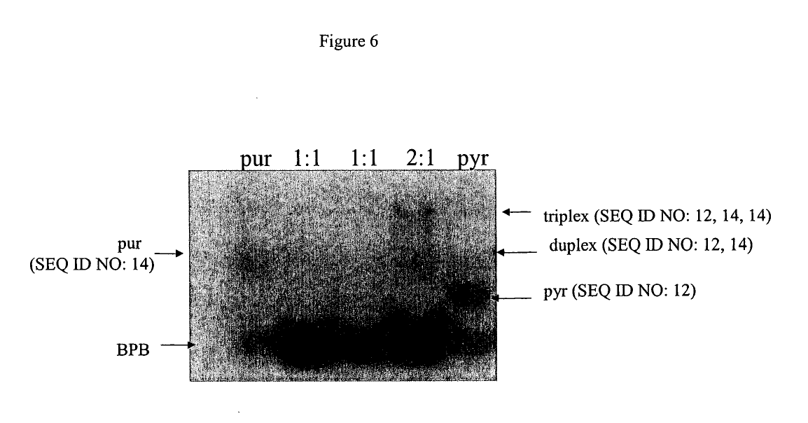

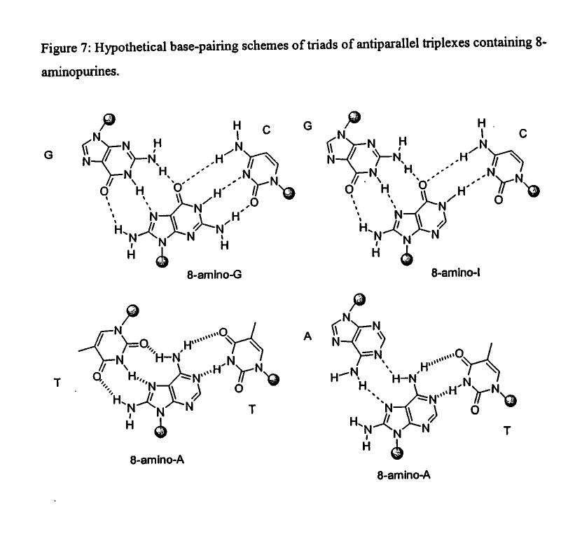

First the effect produced by the presence of 8-aminoguanine in antiparallel triplexes was analyzed on a short intermolecular triplex described by Pilch et al. Triplexes formed by d(C₃T₄C₃).2[d(GG^{N}G^{N}A₄G^{N}G^{N}G)] (SEQ ID NO: 12, SEQ ID NO: 14, SEQ ID NO: 14) were analyzed by melting and gel-shift experiments. The stoichiometry associated with the interaction of these two oligonucleotides was determined by PAGE retardation assay. Mixtures containing 1:1 and 1:2 stoichiometric ratios of d(C₃T₄C₃) (SEQ ID NO: 12) and d(GG^{N}G^{N}A₄G^{N}G^{N}G) (SEQ ID NO: 14) were separated by PAGE. Results are shown in FIG. 6. Only a single band, corresponding to the duplex, is present at 1:1 ratio. This band has a similar mobility than the purine strand. At 1:2 ratio a new band is observed with reduced mobility that corresponds to the triplex.

Thermal denaturation curves of both d(C₃T₄C₃).d(GG^{N}G^{N}A₄G^{N}G^{N}G) duplex (SEQ ID NO: 12, SEQ ID NO: 14) and d(C₃T₄C₃).2[d(GG^{N}G^{N}A₄G^{N}G^{N}G)] triplex (SEQ ID NO: 12, SEQ ID NO: 14, SEQ ID NO: 14) were determined spectrophotometrically at 260 nm in 50 mM MgCl₂, 10 mM sodium cacodylate, 0.1 mM EDTA at pH 7.2. In these conditions a single is observed for both duplex and triplex. The dependence of the melting temperature on DNA concentration was studied. In all cases the plot of 1/Tm versus the concentration was linear, giving a slope and a y-intercept from which ΔH, ΔS, and ΔG were obtained. Table 6 summarizes the thermodynamic parameters obtained. The substitution of four guanines for 8-aminoguanines changes ΔG of duplex to random coil transition from −12.6 kcal/mol to −10 kcal/mol (a decrease of 2.6 kcal/mol). On the contrary, the same substitution changes ΔG of triplex to random coil transition from −26.3 kcal/mol to −28.4 kcal/mol (an increase of 2.1 kcal/mol). Combination of these numbers shows that the presence of four G^{N} stabilizes the triplex (with respect to the duplex) 4.7 kcal/mol. Assuming that the effect of G^{N} is fully additive, stabilization of around 1.2 kcal/mol x substitution, which compares very well with the MD/TI estimate (1.5 kcal/mol from Table 5). That is, the G→G^{N} substitution in the Watson-Crick position strongly stabilize antiparallel triplexes containing the d(G-G•C) triad.

Thermal Stability of Triplexes Formed by from Reverse Watson-Crick Hairpins.

An interesting alternative to create triplexes taking advantage of the stabilizing effect of 8-aminopurines in the Watson-Crick position consists of the use of duplexes to target single stranded nucleic acids, instead of the usual approach of targeting duplexes with single stranded DNA. Parallel-stranded hairpins carrying 8-aminopurines have been proven to form very stable parallel triplexes, opening the possibility of using triplex strategies in the antisense world to target single stranded RNAs. In addition to their excellent binding properties, the use of hairpins as templates for triplex formation present several potential advantages including better nuclease resistance, and unlimited possibilities of functionalization.

In this context, we wanted to analyze whether or not oligonucleotides carrying 8-aminopurines, and designed to have the possibility to form antiparallel reverse Hoogsten hairpins can be templates for antiparallel triplex formation. The effect of 8-aminoguanine, 8-aminoadenine, and 8-aminohypoxanthine (I^{N}) in the triplex forming properties of reverse Hoogsteen hairpins was studied. Oligonucleotides used in the study are shown in Table 9. The parent polypurine-polypyrimidine sequences (H26GA (SEQ ID NO: 16, SEQ ID NO: 17) and H26GT (SEQ ID NO: 16, SEQ ID NO: 18)) were taken from a parallel triplex, where the linking between the Watson-Crick polypurine and the reverse Hoogsteen strands was done by a tetrathymidine loop. Note that these hairpins are designed to form antiparallel triplexes with the polypyrimidine sequence WC-11mer (⁵′TCTCCTCCTTC³′) (SEQ ID NO: 15). 8-Aminopurine derivatives were introduced in different positions of the Watson-Crick strand of the putative hairpin. Thus, in oligonucleotides H26GA(2A^{N}) (SEQ ID NO: 19) and H26GT(2A^{N}) (SEQ ID NO: 18) two adenines were replaced by two 8-aminoadenines; in oligonucleotides H26GA(2G^{N}) (SEQ ID NO: 21) and H26GT(2G^{N}) (SEQ ID NO: 20) two guanines were replaced by two 8-aminoguanines and in oligonucleotides H26GA(2I^{N}) (SEQ ID NO: 23) and H26GT(2I^{N}) (SEQ ID NO: 22) two guanines were replaced by two 8-aminohypoxanthines. The corresponding oligonucleotides carrying two hypoxanthines H26GA(2I) (SEQ ID NO: 27) and H26GT(2I) (SEQ ID NO: 26) were also prepared for comparison. Finally oligonucleotides H26GT(5A^{N}) (SEQ ID NO: 24) and H26GA(6G^{N}) (SEQ ID NO: 25) contained all adenines and guanines at Watson-Crick positions replaced by 8-aminoadenines and 8-aminoguanines respectively. Control oligonucleotides with a scrambled Hoogsteen strand (H26contGT (SEQ ID NO: 28) and H26contGA (SEQ ID NO: 29)) and without the reverse Hoogsteen strand were also prepared (S11pur (SEQ ID NO: 30), S11pur2A^{N} (SEQ ID NO: 31), S11pur2G^{N} (SEQ ID NO: 32).

The relative stability of triple helices formed by H26GA (SEQ ID NO: 17) and H26GT (SEQ ID NO: 16) hairpins and polypyrimidine target sequence (WC-11mer (SEQ ID NO: 15)) was measured spectrophotometrically at 260 nm in 50 mM MgCl₂, pH 7.2. In all cases, one single transition characterized as a transition from triple helix to random coil was observed with 15% hyperchromicity. Monophasic curves were only observed when H26GA (SEQ ID NO: 17) and H26GT (SEQ ID NO: 16) hairpins were mixed with the polypyrimidine target sequence (WC-11mer (SEQ ID NO: 15)). This finding strongly suggests that H26GA (SEQ ID NO: 17) and H26GT (SEQ ID NO: 16) are not fully preorganized before the binding to the third strand, in clear contrast with the behavior found for Hoogsteen hairpins, which were fully organized even in the absence of the Watson-Crick polypyrimidine strand, as demonstrated by MD, CD and NMR data.

Triplexes obtained by incubation of hairpins H26GA (SEQ ID NO: 17), H26GT (SEQ ID NO: 16) and their derivatives with the polypyrimidine oligonucleotide d(TCTCCTCCTTC) (SEQ ID NO: 15) showed melting temperatures in the range from about 54-77° C. (Table 7). That is to say, corresponding triplexes are very stable at physiological temperatures. Interestingly, the control duplex formed by WC-111mer (SEQ ID NO: 15) and the corresponding polypurine strand (without the reverse Hoogsteen strand, S11 derivatives) melted at lower temperatures (about from 42-50° C.). The addition of a non-sense Hoogsteen strand (H26contGT (SEQ ID NO: 28) and H26contGA (SEQ ID NO: 29)) gave similar melting temperatures than control duplexes without Hoogsteen strand (S11 derivatives), demonstrating the specificity of the triplex formation.

As described above and predicted by MD and MD/TI calculations replacement of guanine by 8-aminoguanines stabilizes triple helices. This is confirmed again in an increase on melting temperature of the triplex between from about 2-4° C. per substitution (H26GT2AG (SEQ ID NO: 20) ΔTm/substitution +3.7° C., H26GA2AG (SEQ ID NO: 21) ΔTm/substitution +4.1° C. and H26GA6AG (SEQ ID NO: 25) ΔTm/substitution +2.4° C.). Replacement of adenine by 8-aminoadenine in the H26GT (SEQ ID NO: 16) hairpin stabilizes the triplex around 1° C. per substitution (H26GT2AA (SEQ ID NO: 18) ΔTm/substitution +0.6° C. and H26GT5AA (SEQ ID NO: 24) ΔTm/substitution +1.2° C.), suggesting that, as predicted from MD and MD/TI calculations, the A→A^{N} substitution stabilizes the d(T-A•T) triads. It is worthwhile to analyze the results obtained by the introduction of 8-aminoadenines in the H26GA (SEQ ID NO: 17) hairpin, since in this case the triads involving adenines should be d(A-A•T). For this type of triad, simple molecular models (see FIG. 1) predict that the triplex should be strongly destabilized by the presence of A^{N} in the Watson-Crick position due to strong amino-amino interactions (test MD simulations lead to opened triads). While this invention is not bound by any particular theory, experimental data in Table 7 confirms this prediction (H26GT2I (SEQ ID NO: 27) ΔTm/substitution −3.8° C., and H26GA2I ΔTm/substitution −4.8° C.), strongly suggesting that the effect of 8-aminopurine in the melting curves is due to specific (d(G-G•C) and d(T-A•T)) triad stabilization, as predicted by simulations, and not by an unspecific aggregation effect.

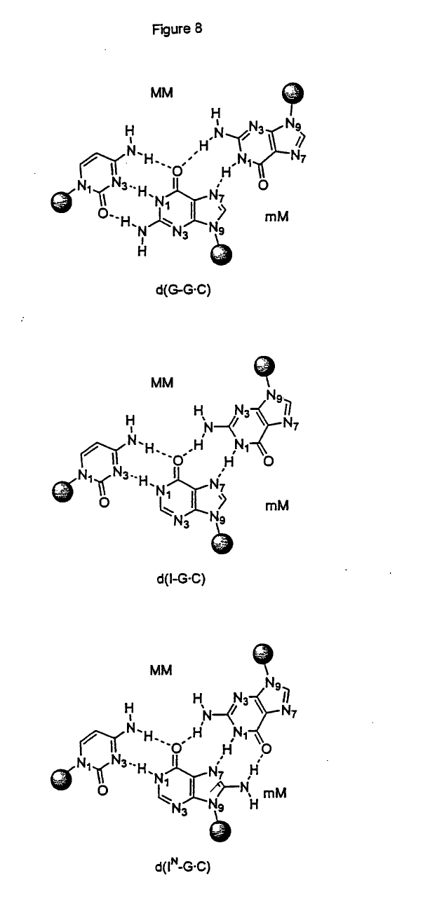

In another embodiment of the present invention, we have analyzed experimentally the effect of the substitution of guanine by hypoxanthine. If the triplex models explained above are correct this should produce a strong destabilization in the triplex due to the loss of one hydrogen bond in the Watson-Crick pair, and this is the result found in Table 7 (H26GT2I (SEQ ID NO: 26) ΔTm/substitution −3.8° C., and H26GA2I (SEQ ID NO: 27) ΔTm/substitution −4.8° C.). Following the same reasoning, the substitution of hypoxanthine by 8-aminohypoxanthine is expected to recover part of the stability, since the lost of one Watson-Crick hydrogen bond is at partially compensated by a new hydrogen bond in the reverse Hoogsteen pair (FIG. 8). We have found that experimental data fully agrees with these predictions.

Thermodynamic Studies

The dependence of the triplex to random coil transition on DNA concentration was studied on several triplexes. In all cases, the melting temperatures of the transitions decrease with the concentration, as expected for a bimolecular transition. The plot of 1/Tm versus ln concentration was linear, giving a slope and a y-intercept from which ΔH, ΔS and ΔG were obtained. Results displayed in Table 8 show that the ΔG for the triplex dissociation were −17.1 and −18.2 kcal/mol for the unmodified H26GT: WC11mer (SEQ ID NO: 16, SEQ ID NO: 15) and H26GA: WC11mer (SEQ ID NO: 17, SEQ ID NO: 15) triplexes. The substitution of two adenines by two 8-aminoadenines in the H26GT2AA: WC11mer (SEQ ID NO: 18, SEQ ID NO: 15) triplex gave a difference in ΔG of 4.6 kcal/mol. Assuming that the stabilizing of A^{N} is additive this would imply a stabilization of the triplex by 2.3 kcal/mol per substitution (MD/TI calculations shown in Table 5 suggested a stabilization of 2.6 kcal/mol). As expected, the same substitution in the reverse Hoogsteen position produce a destabilization of 0.7 Kcal/mol (0.35 kcal/mol per substitution). Triplexes carrying G^{N} gave stabilization in ΔG of 6.7 kcal/mol (3.3 kcal/mol per substitution) and 3.7 kcal/mol (1.8 kcal/mol per substitution), values which compare well with the theoretical estimate of 2.1 kcal/mol in Table 5. This and previous quantitative agreements between theory and experiment confirms the utility of “state of the art” molecular simulation as a quantitative tool for predicting structure and stability of novel nucleic acid structures.

Overall, the bulk of theoretical and experimental data set forth herein not only helps to understand the characteristics of antiparallel triplexes, but also shows the excellent binding properties of hairpins carrying 8-aminopurines to polypyrimidine targets by formation of purine-purine-pyrimidine antiparallel triplexes. The enhancement in binding properties of the modified hairpins yields sequence-specific triplexes very stable at room temperature. This, coupled with the lack of an acidic pH requirement, strongly suggests that the hairpins of the present invention described herein have unique potential use in applications involving oligonucleotide targeting of single stranded RNA or DNA in vitro and in vivo.

The transitions followed experimentally here are slightly different to those computed in our simulations, since experimental values include both Watson-Crick duplex formation and triplex formation from this duplex. The effect of 8-aminopurines in WC duplex stability is always unfavorable (in most cases between a few tenths of kcal/mol) in other cases larger values can be obtained. Accordingly, data obtained in this section can be safely used to discuss triplex stability (vs. Watson-Crick duplex or vs. single stranded DNAs), but quantitative comparison with theoretical values must be taken with caution.

Structural Data from NMR Experiments

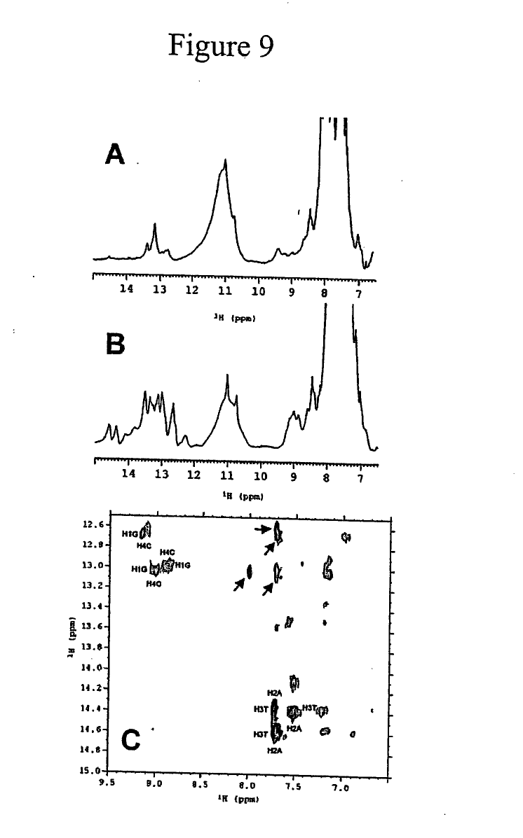

Exchangeable proton NMR spectra of the triplex formed by H26GT (SEQ ID NO: 16) H26GT2AG (SEQ ID NO: 20) with their polypyrimidine target WC-11mer (SEQ ID NO: 15) are shown in FIG. 9. In the case of the unmodified triplex, the signals are rather broad, indicating that some conformational or solvent exchange is taking place. This dynamic effect is clearly reduced in the case of the modified triplex, where the signals are much narrower. In both cases the large signal line widths prevent the acquisition of two-dimensional spectra of enough quality for a complete sequential assignment of the proton spectra. However, some key resonances could be identified as shown in FIG. 9C.

Chemical shifts of the exchangeable protons signals as well as NOESY cross-peaks clearly show that H26GT2AG (SEQ ID NO: 20) hybridizes with WC-11mer (SEQ ID NO: 15), forming an antiparallel triplex. At least three NOE cross-peaks between the imino protons, with chemical shifts between 14 and 15 ppm, and adenines H2 protons are observed, indicating the formation of A-T Watson-Crick base pairs. Also, cross-peaks between guanine iminos and some cytosine amino protons were found, showing the occurrence of G-C Watson-Crick base pairs. In addition, some imino protons, with chemical shifts between 12 and 14 ppm, present intense cross-peaks with non-exchangeable base protons. These protons were identified in the D20 spectra H8 of adenines or guanines, and their NOEs with iminos are indicative of the formation of Hoogsteen G-G or T-A base pairs. In summary, NMR data clearly confirm the existence of antiparallel triple helices, and the gain in structure obtained by the introduction of 8-aminopurine derivatives.

An improved antiparallel triplex is provided, the improvement comprising the substitution of at least one purine in the triplex with at least one 8-aminopurine.

An oligonucleotide hairpin is provided, comprising a first oligonucleotide strand, a linker, and a second oligonucleotide strand, wherein the first oligonucleotide strand is substantially a purine strand comprising at least one 8-aminopurine, and the linker is connected to either the 3′ end of the first oligonucleotide strand and the 5′ end of the second oligonucleotide strand or to the 5′ end of the first oligonucleotide strand and the 3′ end of the second oligonucleotide strand.

An oligonucleotide hairpin is provided, comprising a first oligonucleotide strand, a linker, and a second oligonucleotide strand, wherein the first oligonucleotide strand is substantially a purine strand comprising at least one 8-aminopurine, the linker is connected to either the 3′ end of the first oligonucleotide strand and the 5′ end of the second oligonucleotide strand or to the 5′ end of the first oligonucleotide strand and the 3′ end of the second oligonucleotide strand. Preferably, the 8-aminopurine is selected from 8-aminoadenine, 8-aminoguanine, and 8-aminohypoxanthine.

An oligonucleotide hairpin is provided, comprising a first oligonucleotide strand, a linker, and a second oligonucleotide strand, wherein the first oligonucleotide strand is substantially a purine strand comprising at least one 8-aminopurine, and the linker is a tetrathymine linker connected to either the 3′ end of the first oligonucleotide strand and the 5′ end of the second oligonucleotide strand or to the 5′ end of the first oligonucleotide strand and the 3′ end of the second oligonucleotide strand.

An oligonucleotide hairpin is provided, comprising a first oligonucleotide strand, a linker, and a second oligonucleotide strand, wherein the first oligonucleotide strand is substantially a purine strand comprising at least one 8-aminopurine, the linker is connected to either the 3′ end of the first oligonucleotide strand and the 5′ end of the second oligonucleotide strand or to the 5′ end of the first oligonucleotide strand and the 3′ end of the second oligonucleotide strand, and the second oligonucleotide strand comprises guanine and adenine.

An oligonucleotide hairpin is provided, comprising a first oligonucleotide strand, a linker, and a second oligonucleotide strand, wherein the first oligonucleotide strand is substantially a purine strand comprising at least one 8-aminopurine, the linker is connected to either the 3′ end of the first oligonucleotide strand and the 5′ end of the second oligonucleotide strand or to the 5′ end of the first oligonucleotide strand and the 3′ end of the second oligonucleotide strand, and the second oligonucleotide strand comprises guanine and thymine.

An oligonucleotide hairpin is provided, comprising a first oligonucleotide strand, a linker, and a second oligonucleotide strand, wherein the first oligonucleotide strand is substantially a purine strand comprising at least one 8-aminopurine, the linker is connected to either the 3′ end of the first oligonucleotide strand and the 5′ end of the second oligonucleotide strand or to the 5′ end of the first oligonucleotide strand and the 3′ end of the second oligonucleotide strand. The first oligonucleotide strand is substantially complementary to a target oligonucleotide.

The invention provides an oligonucleotide duplex comprising a first oligonucleotide strand and a second oligonucleotide strand, wherein the first oligonucleotide strand is substantially a purine strand comprising at least one 8-aminopurine and the second oligonucleotide strand is substantially complementary to and chemically bound to the first oligonucleotide strand.

A method for stabilizing an antiparallel oligonucleotide triplex is provided, including the steps of providing an antiparallel oligonucleotide triplex comprising a first, second, and third oligonucleotide strand, wherein at least one oligonucleotide strand comprises a purine, and replacing that purine with an 8-aminopurine.

The invention provides an antiparallel triplex, comprising a first oligonucleotide strand comprising at least one 8-aminopurine, a linker connected to the first strand, a second oligonucleotide strand connected to the opposite end of the linker from the first oligonucleotide strand and capable of forming a hairpin with the first oligonucleotide strand, and a third oligonucleotide strand comprising pyrimidines, wherein the third oligonucleotide strand is substantially complementary to and antiparallel to the first oligonucleotide strand.

The invention also includes an antiparallel triplex, comprising a first oligonucleotide strand comprising at least one 8-aminopurine, a linker connected to the first strand, a second oligonucleotide strand connected to the opposite end of the linker from the first oligonucleotide strand and capable of forming a hairpin with the first oligonucleotide strand, and a third oligonucleotide strand comprising pyrimidines, wherein the third oligonucleotide strand is substantially complementary to and antiparallel to the first oligonucleotide strand. The second oligonucleotide is bound to the first oligonucleotide in either a Hoogsteen configuration or a reverse Hoogsteen configuration.

The invention also includes a method for targeting single-stranded DNA or RNA of a sample, in vivo or in vitro, comprising introducing an oligonucleotide hairpin having at least one 8-aminopurine substitution to a sample solution, the sample solution optionally comprising a target single-stranded DNA or RNA, and the oligonucleotide hairpin capable of forming an antiparallel triplex with the single-stranded DNA or RNA. The sample solution may have a neutral, basic, or acidic pH.

TABLE 1

Summary of calculations done with parallel triplexes containing
normal and 8-aminoderivatives. Simulations with duplexes containing
the 8-amino derivatives are also displayed for completeness.

| | | Length | | | Length |
|---|---|---|---|---|---|
| Oligo | Name | simulation | Oligo | Name | simulation |
| d(GGGGGGGGGG) ¹ | T1 | 5 ns | d(GGGGGGGGGG) ¹ | T1n | 2 ns |
| d(CCCCCCCCCC) ² | | | d(CCCCCCCCCC) ² | | |
| d(GGGGGGGGGG) ¹ | | | d(GGGGG^{N}GGGGG) ⁵ | | |
| d(GGGGG^{N}GGGGG) ⁵ | T1nn | 2 ns | d(CCTCCCTCTC) ¹⁰ | T1d^{a} | 2 ns |
| d(CCCCCCCCCC) ² | | | d(GGAGG^{N}GAGAG) ¹¹ | | |
| d(GGGGG^{N}GGGGG) ⁵ | | | | | |
| d(AAAAAAAAAA) ⁴ | T2 | 3 ns | d(AAAAAAAA) | T5 | 2 ns |
| d(TTTTTTTTTT) ³ | | | d(TTTTTTTT) | | |
| d(AAAAAAAAAA) ⁴ | | | d(AAAAAAAA) | | |
| d(TTTTTTTTTT) ³ | T3 | 3 ns | d(TTTTTTTTT) | T6 | 2 ns |
| d(TTTTTTTTTT) ³ | | | d(TTTTTTTTT) | | |
| d(AAAAAAAAAA) ⁴ | | | d(AAAAAAAAA) | | |
| d(TTTTTTTTT) | T6n | 2 ns | d(TTTTTTTTT) | T6d | 2 ns |
| d(TTTTTTTTT) | | | d(AAAAA^{N}AAAA) | | |
| d(AAAAA^{N}AAAA) | | | | | |
| d(GTGTTTGTTG) ⁶ | T4 | 3 ns | d(GTGTTTGTTG) | T4n | 2 ns |
| d(CTCTTTCTTC) ⁷ | | | d(CTCTTTCTTC) | | |
| d(GAGAAAGAAG) ⁸ | | | d(GAGAA^{N}AGAAG) | | |
| D(CTCTTTCTTC) ⁷ | T4d | 2 ns | | | |
| d(GAGAA^{N}AGAAG) ⁹ | | | | | |
| d(TGGTGGT) | T7a | 2 ns | d(TGGTGGT) | T7b | 2 ns |
| d(TCCTCCT) | | | d(TCCTCCT) | | |
| d(AGGAGGA) | | | d(AGGAGGA) | | |

¹ (SEQ ID NO: 1)
² (SEQ ID NO: 2)
³ (SEQ ID NO: 3)
⁴ (SEQ ID NO: 4)
⁵ (SEQ ID NO: 5)
⁶ (SEQ ID NO: 6)
⁷ (SEQ ID NO: 7)
⁸ (SEQ ID NO: 8)
⁹ (SEQ ID NO: 9)
¹⁰ (SEQ ID NO: 10)
¹¹ (SEQ ID NO: 11)
^{a} Note that the sequence of the duplex matches always that of the parent triplex except for this case, where some adenines replaced guanines to avoid an A-phylic duplex.

TABLE 2

RMSd (in Å) between the
different triplexes studied here and
several reference structures. Standard
deviations (in Å) are shown in parentheses.

| | | | |
|---|---|---|---|
| Triplex | MD-av^{a} | 134D.pdb^{b} | 135D.pdb^{b} |

| | | | |
|---|---|---|---|
| T1 ¹ | 1.7(0.5) | 2.1(0.3) | 2.1(0.4) |
| T1n ² | 1.0(0.4) | | |
| T1nn ³ | 1.1(0.3) | | |
| T2 ⁴ | 1.2(0.3) | 2.1(0.2) | 2.0(0.2) |
| T3 ⁵ | 1.1(0.3) | 2.0(0.1) | 1.7(0.1) |
| T4 ⁶ | 1.5(0.4) | 2.3(0.2) | 2.1(0.2) |
| T4n ⁷ | 1.3(0.3) | | |
| T5 | 1.2(0.3) | 2.0(0.2) | 1.9(0.2) |
| T6 | 1.4(0.3) | 2.2(0.2) | 2.0(0.2) |
| T6n | 1.4(0.3) | | |
| T7a | 0.9(0.2) | 2.1(0.2) | 2.2(0.2) |
| T7b | 1.1(0.2) | 2.1(0.2) | 2.1(0.2) |

¹ (SEQ ID NO: 1, SEQ ID NO: 2, SEQ ID NO: 1)
² (SEQ ID NO: 1, SEQ ID NO: 2, SEQ ID NO: 5)
³ (SEQ ID NO: 5, SEQ ID NO: 2, SEQ ID NO: 5)
⁴ (SEQ ID NO: 4, SEQ ID NO: 3, SEQ ID NO: 4)
⁵ (SEQ ID NO: 3, SEQ ID NO: 3, SEQ ID NO: 4)
⁶ (SEQ ID NO: 6, SEQ ID NO: 7, SEQ ID NO: 8)
⁷ (SEQ ID NO: 6, SEQ ID NO: 7, SEQ ID NO: 9)
^{a} Values computed with respect to the respective MD-averaged conformation using all the atoms in the corresponding oligonucleotide.
^{b} Values computed using only backbone atoms (including C1′) for the common 7-mer sequence.

TABLE 3

Backbone RMSd (in Å) between different
MD-averaged structures of antiparallel triplexes
(T1, T2, T3 and T4) and the MD-averaged structure
of the polyd(T-A·T) parallel triplex in reference
69. Values in roman were obtained by fitting
only the Watson-Crick strands, values in italics
were derived by fitting the three backbones.

| | | | |
|---|---|---|---|
| Parallel | T1 | T2 | T3 |

| | | | | |
|---|---|---|---|---|
| T1 ¹ | 1.7 | — | — | — |
| T2 ⁴ | 1.3 | 0.8/2.2 | — | — |
| T3 ⁵ | 2.3 | 2.2/3.0 | 2.0/2.8 | — |
| T4 ⁶ | 1.5 | 1.3/2.3 | 1.0/2.1 | 1.3/2.2 |

¹ (SEQ ID NO: 1, SEQ ID NO: 2, SEQ ID NO: 1)
⁴ (SEQ ID NO: 4, SEQ ID NO: 3, SEQ ID NO: 4)
⁵ (SEQ ID NO: 3, SEQ ID NO: 3, SEQ ID NO: 4)
⁶ (SEQ ID NO: 6, SEQ ID NO: 7, SEQ ID NO: 8)

TABLE 4

Helical parameters (Watson-Crick strands) of antiparallel
triplexes T1-T4. Angular values are in degrees and displacement
values are in Å. Only the central 8-mer Watson Crick of the triplex
is considered for the analysis. Average NMR data are displayed
for comparison (roman data from pdb134d and italics from pdb135d).

| | | | | |
|---|---|---|---|---|
| T1 ¹ | T2 ⁴ | T3 ⁵ | T4 ⁶ | NMR |

| | | | | | | | | | |
|---|---|---|---|---|---|---|---|---|---|
| X-disp | −2.7 | (0.7) | −4.9 | (1) | −3.2 | (2) | −4.8 | (2) | −2.1/−1.9 |
| Inclination | −3.3 | (6) | 10.9 | (7) | −0.1 | (5) | 11.6 | (14) | −0.4/−4.8 |
| Rise | 3.4 | (0.1) | 3.4 | (0.2) | 3.4 | (0.1) | 3.4 | (0.1) | 3.6/3.7 |
| Roll | 2.9 | (2) | 0.8 | (2) | −3.9 | (1) | −0.5 | (2) | 1.8/−0.6 |
| Twist | 30.1 | (1) | 30.3 | (0.9) | 32.1 | (0.7) | 30.5 | (1) | 30.0/30.3 |
| Prop. Twist | −2.1 | (5) | −3.9 | (5) | −8.2 | (4) | −6.0 | (5) | −11/−13 |
| Phase | 131 | (29) | 128 | (32) | 134 | (23) | 131 | (27) | 122/121^{a} |
| mGroove | 12.6 | (0.5) | 12.3 | (0.5) | 10.5 | (0.4) | 11.4 | (0.5) | 12.7/12.2^{a} |
| mM Groove | 10.8 | (0.6) | 10.4 | (0.6) | 11.7 | (0.4) | 11.7 | (0.4) | 9.0/8.8^{a} |
| MM groove | 12.8 | (2.5) | 12.9 | (0.9) | 12.9 | (0.7) | 12.5 | (1) | 14.5/14.7^{a} |

¹ (SEQ ID NO: 1, SEQ ID NO: 2, SEQ ID NO: 1)
⁴ (SEQ ID NO: 4, SEQ ID NO: 3, SEQ ID NO: 4)
⁵ (SEQ ID NO: 3, SEQ ID NO: 3, SEQ ID NO: 4)
⁶ (SEQ ID NO: 6, SEQ ID NO: 7, SEQ ID NO: 8)
^{a}NMR data is obtained for a 7-mer oligonucleotide which implies large uncertainties in the definition of the grooves.

TABLE 5

Free energy changes in the
triplex→duplex transition induced by changes
from purine to 8-amino purine (a positive
sign implies stabilization of the triplex).
Standard errors are shown in parentheses.

| | | |
|---|---|---|
| Mutation | Triplexes | ΔΔ(kcal/mol) |

| | | |
|---|---|---|
| G→G^{N} | T1n ² | 2.1(0.2) |
| G→G^{N} | T1nn ³ | 1.5(0.2) |
| A→A^{N} | T4n ⁷ | 2.6(0.2) |

¹ (SEQ ID NO: 1, SEQ ID NO: 2, SEQ ID NO: 5)
³ (SEQ ID NO: 5, SEQ ID NO: 2, SEQ ID NO: 5)
⁷ (SEQ ID NO: 6, SEQ ID NO: 7, SEQ ID NO: 9)

TABLE 6

Thermodynamic parameters of the triplex
and the duplex. Data obtained in 10 mM sodium
cacodylate, 50 mM MgCl₂ and 0.1 mM EDTA at pH 7.2.

| | | | | |
|---|---|---|---|---|
| Structure | Tm(° C.) | ΔH (kcal/mol) | ΔS (cal/mol. ° K) | ΔG₂₅ (kcal/mol) |

| unmodified | 52.0 | −72 | −198 | −12.6 |
|---|---|---|---|---|
| duplex^{a} | | | | |
| 8-aminoG | 44.7^{C} | −28 | −63 | −10.0 |
| duplex^{b} | | | | |
| unmodified | 54.0 | −152 | −424 | −26.0 |
| triplex^{a} | | | | |
| 8-aminoG | 53.5^{C} | −133 | −350 | −28.4 |
| triplex^{b} | | | | |
| ^{a}unmodified duplex: d(C₃T₄C_{3).d(G}₃A₄G₃) (SEQ ID NO: 12, SEQ ID NO: 13); unmodified triplex d(C₃T₄C₃).2[d(G₃A₄G₃)] (SEQ ID NO: 12, SEQ ID NO: 13, SEQ ID NO: 13) | | | | |
| ^{b}8-aminoG duplex: d(C₃T₄C₃).d(GG^{N}G^{N}A₄G^{N}G^{N}G) (SEQ ID NO: 12, SEQ ID NO: 14); 8-aminoG triplex d(C₃T₄C₃).2[d(GG^{N}G^{N}A₄G^{N}G^{N}G)].(SEQ ID NO: 12, SEQ ID NO: 14, SEQ ID NO: 14) | | | | |
| ^{C}Tm at a 4 μM concentration | | | | |
| ΔG₂₅ refers to the standard free energy change at 25° C. | | | | |

TABLE 7

Melting temperatures (° C.) for
triplexes formed by H26GA (SEQ ID NO: 15,
SEQ ID NO: 16, SEQ ID NO: 17) and H26GT (SEQ ID
NO: 15, SEQ ID NO: 16, SEQ ID NO: 18) derivatives
and WC-11mer. Data obtained in 10 mM sodium
cacodylate, 50 mM MgCl₂ and 0.1 mM EDTA at pH 7.2.

| | |
|---|---|
| WC-11 mer(SEQ ID NO: 15) | WC-11 mer(SEQ ID NO: 15) |
| 3′-CTTCCTCCTCT | 3′-CTTCCTCCTCT |
| \|\|\|\|\|\|\|\|\|\|\| | \|\|\|\|\|\|\|\|\|\|\| |
| 5′-GAAGGAGGAGA^{T} | 5′-GAAGGAGGAGA^{T} |
| ........... T | ........... T |
| ........... T | ........... T |
| 3′-GAAGGAGGAGA_{T} | 3′-GTTGGTGGTGT_{T} |
| H26GA | H26GT |
| (SEQ ID NO: 16) | (SEQ ID NO: 17) |

| | Tm | | ΔTm/sub- | Duplex | Duplex |
|---|---|---|---|---|---|
| Hairpin | (° C.)^{a} | ΔTm^{b} | stitution | (S11) | (H26con) |
| H26GT¹ | 60.5 | — | — | 50.0^{c} | 51.0^{g} |
| H26GT(2A^{N})² | 61.7 | +1.2 | +0.6 | 45.6^{d} | — |
| H26GT(2G^{N})³ | 68.0 | +7.5 | +3.7 | 41.1^{e} | — |
| H26GT(2I^{N})⁴ | 61.5 | +1.0 | +0.5 | — | — |
| H26GT(2I)⁵ | 52.8 | −7.7 | −3.8 | 38.5^{f} | — |
| H26GT(5A^{N})⁶ | 66.6 | +6.1 | +1.2 | — | — |
| H26(GA)⁷ | 62.7 | — | — | 50.0^{c} | 50.0^{h} |
| H26GA(2A^{N})⁸ | 56.5 | −6.2 | −3.1 | 45.6^{d} | — |
| H26GA(2G^{N})⁹ | 71.0 | +8.3 | −4.1 | 41.1^{e} | — |
| H26GA(2I^{N})¹⁰ | 62.4 | −0.3 | −0.1 | — | — |
| H26GA(2I)¹¹ | 53.1 | −9.6 | −4.8 | 38.5^{f} | — |
| H26GA(6G^{N})¹² | 77.2 | +4.5 | +2.4 | — | — |

¹(SEQ ID NO: 16);
²(SEQ ID NO: 18);
³(SEQ ID NO: 20);
⁴(SEQ ID NO: 22);
⁵(SEQ ID NO: 26);
⁶(SEQ ID NO: 24);
⁷(SEQ ID NO: 17);
⁸(SEQ ID NO: 19)
⁹(SEQ ID NO: 21);
¹⁰(SEQ ID NO: 23);
¹¹(SEQ ID NO: 27);
¹²(SEQ ID NO: 25)
^{a}at a concentration approx. μM,
^{b}ΔTm = Tm − Tm of the corresponding unmodified H26 derivative (H26GT (SEQ ID NO: 16) or H26GA (SEQ ID NO: 17)),
^{c}S11pur: WC11mer control duplex (SEQ ID NO: 30, SEQ ID NO: 15),
^{d}S11pur2AA: WC11mer control duplex (SEQ ID NO: 31, SEQ ID NO: 15),
^{e}S11pur2AG: WC11mer control duplex (SEQ ID NO: 32, SEQ ID NO: 15),
^{f}S11purI: WC11mer control duplex (SEQ ID NO: 33, SEQ ID NO: 15),
^{g}H26contGT: WC11mer control duplex (SEQ ID NO: 28, SEQ ID NO: 15),
^{h}H26contGA: WC11mer control duplex (SEQ ID NO: 29, SEQ ID NO: 15).

TABLE 8
Thermodynamic parameters for triplex to random coil transitions in 10 mM
sodium cacodylate, 50 mM MgCl₂ and 0.1 mM EDTA at pH 7.2 from the slope of the plot
1/Tm versus ln C^{a}.

| | | | ΔS | |
|---|---|---|---|---|
| triplex | Tm (° C.)^{b} | ΔH (kcal/mol) | (kcal/mol K) | ΔG (kcal/mol) |
| H26GBT p+ WC11mer | 60.7 | −83 | −222 | −17.1 |
| H26GT(2^{N}) + WC11mer | 61.5 | −123 | −342 | −21.7 |
| H26GT(2G^{N}) + WC11mer | 67.6 | −101 | −269 | −20.8 |
| H26GT(2I^{N}) + WC11mer | 62.0 | −96 | −260 | −18.8 |
| H26GA + WC11mer | 63.4 | −88 | −235 | −18.2 |
| H26GA(2A^{N}) + WC11mer | 57.4 | −95 | −260 | −17.5 |
| H26GA(2G^{N}) + WC11mer | 72.5 | −122 | −325 | −24.9 |

^{a}ΔH, ΔS, ΔG are given as round number, ΔG is calculated at 25° C., with the assumption that ΔH and ΔS do not depend on temperature; analysis has been carried out using melting temperaturtes obtained from denaturation curves.
^{b}at 4 μM triplex concentration

TABLE 9

Sequences of
oligonucleotides carrying 8-aminopurines
as prepared in this study; G^{N}, 8-aminoguanine;
A^{N}, 8-aminoadenine; and I^{N}, 8-aminohypoxanthine.

| | |
|---|---|
| H26GT: | |
| ^{5′}GAAGGAGGAGA-TTTT-TGTGGTGGTTG^{3′} | (SEQ ID NO: 16) |
| H26GA: | |
| ^{5′}GAAGGAGGAGA-TTTT-AGAGGAGGAAG^{3′} | (SEQ ID NO: 17) |
| H26GT2AA: | |
| ^{5′}GAAGGA^{N}GGA^{N}GA-TTTT-TGTGGTGGTTG^{3′} | (SEQ ID NO: 18) |
| H26GA2AA: | |
| ^{5′}GAAGGA^{N}GGA^{N}GA-TTTT-AGAGGAGGAAG^{3′} | (SEQ ID NO: 19) |
| H26GT2AG: | |
| ^{5′}GAAGG^{N}AGG^{N}AGA-TTTT-TGTGGTGGTTG^{3′} | (SEQ ID NO: 20) |
| H26GA2AG: | |
| ^{5′}GAAGG^{N}AGG^{N}AGA-TTTT-AGAGGAGGAAG^{3′} | (SEQ ID NO: 21) |
| H26GT2AI: | |
| ^{5′}GAAGI^{N}AGI^{N}AGA-TTTT-TGTGGTGGTTG^{3′} | (SEQ ID NO: 22) |
| H26GA2AI: | |
| ^{5′}GAAGI^{N}AGI^{N}AGA-TTTT-AGAGGAGGAAG^{3′} | (SEQ ID NO: 23) |
| H26GT5AA: | |
| ^{5′}GA^{N}A^{N}GGA^{N}GGA^{N}GA^{N}-TTTT- | (SEQ ID NO: 24) |
| TGTGGTGGTTG^{3′} | |
| H26GA6AG: | |
| ^{5′}G^{N}AAG^{N}G^{N}AG^{N}G^{N}AG^{N}A-TTTT- | (SEQ ID NO: 25) |
| AGAGGAGGAAG^{3′} | |
| H26GT21: | |
| ^{5′}GAAGIAGIAGA-TTTT-TGTGGTGGTTG^{3′} | (SEQ ID NO: 26) |
| H26GA2I: | |
| ^{5′}GAAGIAGIAGA-TTTT-AGAGGAGGAAG^{3′} | (SEQ ID NO: 27) |
| H26contGT: | |
| ^{5′}GAAGGAGGAGA-TTTT-GTGTGGTTTGT^{3′} | (SEQ ID NO: 28) |
| H26contGA: | |
| ^{5′}GAAGGAGGAGA-TTTT-GAGAGGAAAGA^{3′} | (SEQ ID NO: 29) |
| S11pur: | |
| ^{5′}GAAGGAGGAGA^{3′} | (SEQ ID NO: 30) |
| S11pur2AA: | |
| ^{5′}GAAGGA^{N}GGA^{N}GA^{3′} | (SEQ ID NO: 31) |
| S11pur2AG: | |
| ^{5′}GAAGG^{N}AGG^{N}AGA^{3′} | (SEQ ID NO: 32 |
| S11pur2I: | |
| ^{5′}GAAGIAGIAGA^{3′} | (SEQ ID NO: 33) |

Whereas, particular embodiments of this invention have been described for purposes of illustration, it will be evident to those persons skilled in the art that numerous variations of the details of the present invention may be made without departing from the invention as defined in the appended claims.

1. An antiparallel oligonucleotide triplex, the improvement comprising the substitution of at least one purine in said triplex with at least one 8-aminopurine.

2. An oligonucleotide hairpin comprising a first oligonucleotide strand, a linker, and a second oligonucleotide strand, wherein:
(a) said first oligonucleotide strand is substantially a purine strand comprising at least one 8-aminopurine; and
(b) said linker is connected to either the 3′ end of said first oligonucleotide strand and the 5′ end of said second oligonucleotide strand or to the 5′ end of said first oligonucleotide strand and the 3′ end of said second oligonucleotide strand.

3. The oligonucleotide of claim 2, wherein said 8-aminopurine is selected from the group consisting of 8-aminoadenine, 8-aminoguanine, and 8-aminohypoxanthine.

4. The oligonucleotide of claim 2, wherein said linker is tetrathymine.

5. The oligonucleotide of claim 2, wherein said second oligonucleotide strand comprises guanine and adenine.

6. The oligonucleotide of claim 2, wherein said second oligonucleotide strand comprises guanine and thymine.

7. The oligonucleotide of claim 2, wherein said first oligonucleotide strand is substantially complementary to a target oligonucleotide.

8. An oligonucleotide duplex comprising a first oligonucleotide strand and a second oligonucleotide strand, wherein:
(a) said first oligonucleotide strand is substantially a purine strand comprising at least one 8-aminopurine; and
(b) said second oligonucleotide strand is substantially complementary to and chemically bound to said first oligonucleotide strand.

9. A method for stabilizing an antiparallel oligonucleotide triplex, comprising the steps of
(a) providing an antiparallel oligonucleotide triplex comprising a first, second, and third oligonucleotide strand, wherein at least one oligonucleotide strand comprises a purine; and
(b) replacing said purine with an 8-aminopurine.

10. An antiparallel triplex, comprising:
(a) a first oligonucleotide strand comprising at least one 8-aminopurine;
(b) a linker connected to said first strand;
(c) a second oligonucleotide strand connected to the opposite end of said linker from the first oligonucleotide strand and capable of forming a hairpin with said first oligonucleotide strand; and
(d) a third oligonucleotide strand comprising pyrimidines, wherein said third oligonucleotide strand is substantially complementary to and antiparallel to said first oligonucleotide strand.

11. The triplex of claim 10, wherein said second oligonucleotide is bound to said first oligonucleotide in one of a Hoogsteen configuration or a reverse Hoogsteen configuration.

12. A method for targeting single-stranded DNA or RNA of a sample, in vivo or in vitro, comprising introducing an oligonucleotide hairpin having at least one 8-aminopurine substitution to a sample solution, said sample solution optionally comprising a target single-stranded DNA or RNA, said oligonucleotide hairpin capable of forming an antiparallel triplex with said single-stranded DNA or RNA.

13. The method of claim 12, including wherein said sample solution has a neutral, basic, or acidic pH.


SEQUENCE LISTING (33 sequences)

SEQ ID NO: 1 - DNA, 10 bp, Artificial
  synthetic nucleotide sequence
gggggggggg

SEQ ID NO: 2 - DNA, 10 bp, Artificial
  synthetic nucleotide sequence
cccccccccc

SEQ ID NO: 3 - DNA, 10 bp, Artificial
  synthetic nucleotide sequence
tttttttttt

SEQ ID NO: 4 - DNA, 10 bp, Artificial
  synthetic nucleotide sequence
aaaaaaaaaa

SEQ ID NO: 5 - DNA, 10 bp, Artificial
  synthetic nucleotide sequence
ggggnggggg

SEQ ID NO: 6 - DNA, 10 bp, Artificial
  synthetic nucleotide sequence
gtgtttgttg

SEQ ID NO: 7 - DNA, 10 bp, Artificial
  synthetic nucleotide sequence
ctctttcttc

SEQ ID NO: 8 - DNA, 10 bp, Artificial
  synthetic nucleotide sequence
gagaaagaag

SEQ ID NO: 9 - DNA, 10 bp, Artificial
  synthetic nucleotide sequence
gaganagaag

SEQ ID NO: 10 - DNA, 10 bp, Artificial
  synthetic nucleotide sequence
cctccctctc

SEQ ID NO: 11 - DNA, 10 bp, Artificial
  synthetic nucleotide sequence
ggagngagag

SEQ ID NO: 12 - DNA, 10 bp, Artificial
  synthetic nucleotide sequence
cccttttccc

SEQ ID NO: 13 - DNA, 10 bp, Artificial
  synthetic nucleotide sequence
gggaaaaggg

SEQ ID NO: 14 - DNA, 10 bp, Artificial
  synthetic nucleotide sequence
gnnaaaanng

SEQ ID NO: 15 - DNA, 11 bp, Artificial
  synthetic nucleotide sequence
tctcctccttc

SEQ ID NO: 16 - DNA, 26 bp, Artificial
  synthetic nucleotide sequence
gaaggaggagattttagaggaggaag

SEQ ID NO: 17 - DNA, 26 bp, Artificial
  synthetic nucleotide sequence
gaaggaggagatttttgtggtggttg

SEQ ID NO: 18 - DNA, 26 bp, Artificial
  synthetic nucleotide sequence
gaaggnggngatttttgtggtggttg

SEQ ID NO: 19 - DNA, 26 bp, Artificial
  synthetic nucleotide sequence
gaaggnggngattttagaggaggaag

SEQ ID NO: 20 - DNA, 26 bp, Artificial
  synthetic nucleotide sequence
gaagnagnagatttttgtggtggttg

SEQ ID NO: 21 - DNA, 26 bp, Artificial
  synthetic nucleotide sequence
gaagnagnagattttagaggaggaag

SEQ ID NO: 22 - DNA, 26 bp, Artificial
  synthetic nucleotide sequence
gaagnagnagatttttgtggtggttg

SEQ ID NO: 23 - DNA, 26 bp, Artificial
  synthetic nucleotide sequence
gaagnagnagattttagaggaggaag

SEQ ID NO: 24 - DNA, 26 bp, Artificial
  synthetic nucleotide sequence
gnnggnggngntttttgtggtggttg

SEQ ID NO: 25 - DNA, 26 bp, Artificial
  synthetic nucleotide sequence
naannannanattttagaggaggaag

SEQ ID NO: 26 - DNA, 26 bp, Artificial
  synthetic nucleotide sequence
gaagnagnagatttttgtggtggttg

SEQ ID NO: 27 - DNA, 26 bp, Artificial
  synthetic nucleotide sequence
gaagnagnagattttagaggaggaag

SEQ ID NO: 28 - DNA, 26 bp, Artificial
  synthetic nucleotide sequence
gaaggaggagattttgtgtggtttgt

SEQ ID NO: 29 - DNA, 26 bp, Artificial
  synthetic nucleotide sequence
gaaggaggagattttgagaggaaaga

SEQ ID NO: 30 - DNA, 11 bp, Artificial
  synthetic nucleotide sequence
gaaggaggaga

SEQ ID NO: 31 - DNA, 11 bp, Artificial
  synthetic nucleotide sequence
gaaggnggnga

SEQ ID NO: 32 - DNA, 11 bp, Artificial
  synthetic nucleotide sequence
gaagnagnaga

SEQ ID NO: 33 - DNA, 11 bp, Artificial
  synthetic nucleotide sequence
gaagnagnaga